(19) United States
(12) Patent Application Publication
(10) Pub. No.: US 2010/0158817 A1
(43) Pub. Date: Jun. 24, 2010

(54) T1 MRI TRACKABLE DRUG DELIVERY PARTICLES, USES AND METHODS THEREOF

(75) Inventors: Sigrid L. Fossheim, Oslo (NO); Esben A, Nilssen, Olso (NO)

Correspondence Address: BIRCH STEWART KOLASCH & BIRCH, PO BOX 747, FALLS CHURCH, VA, 22040-0747, US

(73) Assignee: EPITARGET AS

(21) Appl. No.: 12/441,116

(22) PCT Filed: Sep. 21, 2007
(86) PCT No.: PCT/NO07/00334

§ 371 Date: Mar. 12, 2009

(30) Foreign Application Priority Data
Sep. 22, 2006 (NO) 20064315

(51) Int. Cl. A61K 49/18; A61K 49/06; A61P 35/04; A61P 9/00; A61P 29/00; A61P 37/00

(52) U.S. Cl. 424/9.321; 424/9.3

(57) ABSTRACT

The current invention discloses a drug delivery system allowing monitoring of spatial position and drug release, as well as methods and uses thereof. More particularly, the drug delivery system comprises drug carrying particles comprising an internal and external distribution of magnetic resonance imaging contrast agents.

### FIELD OF THE INVENTION

The present invention relates to a drug delivery particle allowing monitoring of spatial position and drug release. More particularly, the invention relates to drug carrying particles comprising magnetic resonance imaging contrast agents, as well as methods and uses thereof.

### BACKGROUND OF THE INVENTION

A serious limitation of traditional medical treatment is lack of specificity, that is, drugs do not target the diseased area specifically, but affect essentially all tissues. This limitation is particularly evident in chemotherapy where all dividing cells are affected is imposing limitations on therapy. One strategy to achieve improved drug specificity is incorporation or encapsulation of drugs for example in liposomes, plurogels and polymer particles. To further improve efficiency ultrasound (US) mediated drug release from such particles has been disclosed in several publications, for a review see Pitt et al. (2004). Other approaches are heat mediated release and light mediated release. All these techniques show promise in laboratory or early preclinical studies, but the clinical value is yet to be determined. One challenge in this regard is to monitor both accumulation of the drug delivery entity in the diseased area and the extent of drug release.

Magnetic Resonance Imaging (MRI) is an imaging method routinely used in medical diagnostics. The method is based on interactions between radio waves and body tissue water protons in a magnetic field. The signal intensity of a given tissue is dependent on several factors including proton density, spin lattice (T1) and spin spin (T2) relaxation times of tissue water protons. Tissues with shortened T2 will typically appear as an area of low signal intensity on standard T1 or/and T2 (T2*) weighted MR images whilst tissues with shortened T1 will be visualized on standard T1 weighted MR images as an area of high signal intensity.

Contrast agents are used in imaging to increase the signal intensity difference between the area of interest and background tissue thus enhancing the contrast. In MRI, an increase in signal intensity difference between two tissues is attained by the ability of the contrast agent to selectively shorten the T1 and/or T2 of water protons in a given tissue relative to another. The efficiency of an MRI contrast agent to shorten the T1 and T2 of water protons is defined as the T1 and T2 relaxivity (r1 and r2), respectively. The higher the relaxivity the more efficient is the agent in shortening the relaxation times of water protons.

Several classes of MRI contrast agents exist, the classification depending on their clinical applications, relaxation and magnetic properties. With respect to magnetic properties, one distinguishes between paramagnetic and superparamagnetic agents. Paramagnetic agents are typically based on the lanthanide metal ions, gadolinium (Gd³⁺), dysprosium (Dy³⁺) and the transition metal ions, manganese.(Mn²⁺ and Mn³⁺) and iron (Fe²⁺, Fe³⁺). Due to toxicity, these paramagnetic metal ions need to be administered in the form of stable chelates or other stabilizing entities.

Stabilizing entities may be particulate carriers such as liposomes. Liposomes are spherical colloidal particles consisting of one or more phospholipid bilayers that enclose an aqueous interior. Encapsulation of material in the aqueous interior or incorporation into the phospholipid bilayer provides a means to alter the biodistribution of material and to achieve concentration-time exposure profiles in target tissues that are not readily accomplished with free, i.e. non-liposomal material. Also, the use of sterically stabilised and/or ligand targeted liposome delivery has opened the way for more attractive medical applications, such as medical treatment of tumours and inflammation sites. Some examples of marketed parenteral liposomal drug formulations are: Ambisome®, containing amphotericin B (antifungal agent), Caelyx® containing doxorubicin (chemotherapeutic agent) and DaunoXome® containing daunorubicin (chemotherapeutic agent). Liposomes have also been extensively investigated as carriers for paramagnetic and superparamagnetic materials, but so far no liposomal MRI contrast agents are commercially available.

Liposomes or other particles containing paramagnetic agents shorten the T1 of tissue water protons by so-called dipolar relaxation mechanisms. The latter also contribute to a T2 shortening effect. Another possible contribution to the overall T2 shortening is the susceptibility, also termed T2*, effect of the liposomes. The ability of paramagnetic liposomes to shorten T1 and/or T2(T2*) depends amongst other on the physicochemical properties of both the liposome and paramagnetic agent involved as well as the localization of the latter within the liposome.

For instance, in the case of liposome encapsulated Gd chelate the dipolar T1 relaxation effect is mediated by an exchange process of water molecules between the liposome interior and exterior, i.e. bulk water (Barsky et al. 1994). Depending on the physicochemical properties of the liposome and Gd chelate, the dipolar relaxation effect is either in the slow water exchange or fast water exchange regimes. In simplistic terms, the combination of low liposome permeability and encapsulated Gd agent in sufficiently high amounts will result in an exchange limited dipolar relaxation effect yielding an overall low liposome T1 relaxivity. Various studies have shown how liposome size and composition of the liposome membrane affect the T1 relaxivity of liposome encapsulated Gd agent under conditions of slow water exchange (Tilcock et al. 1989, Fossheim et al. 1999a). Adversely, when the membrane permeability is high enough to relieve any exchange limitations (i.e. fast water exchange regime), the liposomal T1 relaxivity is high and similar to the relaxivity of the free (non-encapsulated) Gd agent (Fossheim et al. 1999a; Fossheim et al. 2000). The same underlying mechanisms apply for dipolar mediated T2 relaxation efficacy of the above systems. However, as long as fast water exchange conditions prevail, liposome encapsulated Gd agent will preferentially act as a T1 agent and increase the signal intensity of a given tissue.

The T1 relaxation properties of a Gd chelate attached to the liposome surface are generally improved as to increase the T1 relaxivity due to a reduced rotational motion of the Gd chelate. The gain in relaxivity however depends on many factors such as the field strength, size of the liposome, membrane permeability and, more importantly, on the type of binding or association between the Gd chelate and liposome surface. High membrane permeability is a prerequisite to exploit the relaxation contribution of the Gd chelate bound to the inner surface of the membrane (i.e. faster water exchange conditions) whilst the relaxation contribution of Gd chelate bound to the outer surface of the liposome is not dependent on membrane permeability. If the binding or association between the liposome surface and Gd chelate is rigid, the motion of the chelate will be modulated by the motion of the larger liposome particle; the larger the size of the liposome the higher will the relaxivity gain be. Up to 10 folds increase in T1 relaxivity has been reported at 0.5 Tesla for Gd chelates upon rigid association to liposomes as compared to non-liposomal Gd chelate (Gløgård et al. 2002). On the other hand, if the Gd chelate exhibits its own rapid motion independent of the larger liposome particle (so-called anisotropic motion prevails), the gain in relaxivity might be small to negligible and the overall relaxivity is size independent (Tilcock et al. 1992). For a comprehensive review on the relaxation mechanisms and properties of Gd chelates see Caravan et al. (1999).

Particulate (e.g. liposomal) paramagnetic agents can also be regarded as a magnetized particle due to the confinement or compartmentalization of a high amount of paramagnetic material within the particle. In such circumstances, long range relaxation mechanisms can develop originating from the magnetic field gradients induced by the difference in magnetic susceptibility between the liposome (containing the agent) and bulk. These long range relaxation mechanisms, are not dependent on water exchange and are usually referred to as susceptibility or T2* effects. Susceptibility effects typically decrease the overall T2 and, hence, signal intensity of a given tissue. In order to maximize the susceptibility effects, paramagnetic materials that have a high magnetic susceptibility are used or more preferably superparamagnetic iron oxides are used.

With respect to paramagnetic susceptibility effects, Dy based compounds are usually preferred materials due to a twice as high magnetic susceptibility than Gd based compounds. Indeed, studies have shown the potential of Dy chelates as susceptibility agents per se or present in particles; no interfering T1 effect will occur due to the very poor dipolar relaxation efficacy of Dy³⁺ ions (Fossheim et al. 1997, 1999b).

In functional terms, a liposome encapsulated Gd agent will preferentially function as a T1 agent when factors such as high membrane permeability favour rapid water exchange between liposome interior and exterior. In cases of low membrane permeability and slow water exchange, liposome encapsulated Gd agent will preferentially act as a T2 or susceptibility (T2*) agent. The same conclusions can be drawn for low permeability liposomes containing Gd agent incorporated or bound to the inner surface of the liposome membrane. A liposome containing outer surface attached Gd chelate will preferentially function as a T1 agent. A liposomal Dy agent will function as a T2 or susceptibility (T2*) agent irrespective of membrane permeability and/or localization within the liposome.

Liposomal formulations containing Gd agents are known from the art.

EP1069888B1, incorporated herein by reference in its entirety, discloses a contrast medium for imaging of a physiological parameter, said medium comprising a matrix or membrane material and at least one magnetic resonance contrast generating species, said matrix or membrane material being responsive to a pre-selected physiological parameter and the response is an increased matrix or membrane permeability or chemical or physical breakdown of the matrix or membrane material, to cause the contrast efficacy of said contrast generating species to vary in response to said parameter. '888B1 does not mention coformulation of drugs and contrast agents. Hence, there is no discussion of drug release and the need to monitor the spatial position, accumulation and concentration of a drug carrying particle, less the need to monitor the efficiency of drug release. In conclusion, no solution to the current problem is disclosed in '888B1.

WO2006/032705 discloses a liposom comprising a paramagnetic chelate, e.g. GdDTPA-MBA, and a drug. A liposome with both an internal and external population of T1 agents is not mentioned or suggested. WO 04/023981 describes so-called envirosensitive liposomes designed to release drugs during specific conditions like high temperature, pH, or acoustic fields. Said liposomes may also comprise a contrast is agent, e.g. gadolinium or dysprosium based materials. None of these inventions may be used for both monitoring position and drug release during e.g. liposomal drug delivery.

Rubesova et al. (2002) describe Gd-labeled liposomes for monitoring liposome-encapsulated chemotherapy. This particle has a high water permeability and displays no water exchange limitations at physiological temperature only making it useful for monitoring spatial position. Hence, the need to concomitantly monitor position, particle concentration and drug release is neither realized nor solved.

Bednarski et al. (1997) report use of liposome encapsulated Gd-DTPA as an MR-detectable model representing pharmaceutical agents. Bednarski et al use liposomal Gd chelate to track the position of the liposome similar to Rubesova supra. Monitoring of drug release is not mentioned and no solution is suggested.

Liposomal membrane bound contrast agents are also known from the art. For a review see Mulder et al. (2006), page 151. However, these liposomes are exclusively used for diagnostic purposes and do not carry drugs.

Also other groups have reported the use of Gd- and Mn loaded liposomes. See Saito et al. (2005), Vigllianti et al. (2004). For a review, see Richardson et al. (2005) and Tilcock (1999).

Hence, the art has so far focused on liposomal formulations of T1 agents, like Gd chelates, for monitoring either position or physiological conditions, i.e. for diagnostic use. Determination of position is dependent on exposure or high accessibility to bulk water, that is, no water exchange limitations, while monitoring of physiological conditions is based on variable water accessibility. The current inventors have realized the need to concomitantly monitor the spatial position, accumulation and concentration of a drug carrying particle, as well as the need to monitor the efficiency of drug release. The present invention is based on the understanding that the above technical problem may be solved by an internal and external distribution of a T1 contrast compound in a robust and stable drug delivery particle. Thus, an MR trackable drug delivery particle allowing monitoring of both spatial coordinates and drug release is disclosed. The invention improves the safety and efficiency of drug delivery from particles, and is particularly useful in ultrasound mediated drug release from particular drug delivery systems.

### Definitions

The use of singular form may herein mean one or several. Hence, ‘a contrast agent’ means one or several contrast agents, unless specified otherwise.

The terms ‘contrast efficiency’ and ‘relaxation efficiency’ are used interchangeably in the current document.

The term ‘internal’ herein means shielded or protected from bulk water up to the point of drug release, i.e. low water accessibility.

The term ‘external’ herein means exposed to bulk water, i.e. high water accessibility.

The term ‘non-physiological parameters’ means physical and chemical parameters not encountered in healthy or diseased mammals. A temperature of 50° C. is an example of a non-physiological parameter.

‘Breakdown’ means both chemical and/or physical breakdown. Physical breakdown includes disruption or opening of the matrix or membrane, while chemical breakdown includes dramatic increase in membrane or matrix permeability, e.g. by pore formation. The breakdown may be both temporary and permanent. In functional terms ‘breakdown’ means release of the carried drug and enhanced overall relaxation enhancement.

T2* effect means susceptibility effect that contributes to the overall T1 shortening in compartmentalized systems.

A ‘contrast agent per se’ means herein any compound with the ability to generate an MRI contrast given the right conditions. The term ‘contrast agent’ may be any contrast compound, contrast generating aggregate, contrast agent per se, contrast generating particle or entity.

The term ‘bulk water’ means herein the water compartment exterior to the particle where the majority of water molecules reside.

### DETAILED DESCRIPTION OF THE INVENTION

The current invention comprises a trackable particulate material for drug delivery comprising a matrix or membrane material, a drug, internal T1 magnetic resonance contrast agents and an external T1 magnetic resonance contrast agent, wherein the relaxation efficacy of the internal T1 species is optimal during and/or after drug release.

More specifically, the current invention comprises trackable particulate material for drug delivery comprising a matrix or membrane material, a drug, internal T1 magnetic resonance contrast agents and an external T1 magnetic resonance contrast agent, wherein the internal T1 agents are shielded from bulk water and the external T1 agent is exposed to bulk water.

Even more particularly, the current invention comprises a trackable particulate material for drug delivery comprising a matrix or membrane material, a drug, and an internal and an external T1 magnetic resonance contrast generating species, wherein the relaxation efficiency of the external species is optimal during the entire drug delivery process and the relaxation efficiency of the internal species is optimal as a result of chemical and/or physical breakdown of the matrix or membrane material.

It is a central feature of the current invention that the internal T1 agents exhibit low or essentially no T1 relaxation effect before the membrane material or matrix breakdown, while the T1 relaxation efficiency of the external T1 agent is optimal during the entire drug delivery process (FIG. 1). In other words, the trackable particulate material as a whole yields a stronger T1 contrast as a result of breakdown of the matrix or membrane material and, consequently, coincides with drug release. This feature presupposes that the water permeability of the matrix or membrane does not increase without drug release. The T2* effect of the T1 contrast agent per se may also decrease as a result of drug release.

The external T1 species must be located on the particulate material in such as to expose it to bulk water, for example, partly or completely on the exterior surface of a liposome. The internal T1 species must, on the other hand, be shielded from bulk water until the point of drug release. In e.g. a liposome this would mean within the membrane, on the interior side of the liposome membrane or in the liposome interior aqueous phase, or combinations thereof.

The membrane or matrix material may be any material suitable for the current task, e.g. lipids or polymer substances. Moreover, the membrane or matrix material may be an amphiphilic substance capable of forming a liquid crystalline phase, in contact with a liquid selected from the group consisting of water, glycerol, ethylene glycol, propylene glycol and mixtures thereof. The water permeability of the intact matrix or membrane material must, however, impose relaxation exchange limitations, as described above. That is, the permeability, preferably the water permeability, of the membrane or matrix material must possess characteristics not allowing a high level T1 relaxation efficiency of the internal T1 species. Typically the membrane permeability will be at a level essentially eliminating any T1 relaxation effects of said internal contrast species. It is an essential aspect of the present invention that the membrane or matrix material should be non-responsive vis-à-vis both normal and pathological physiological conditions in terms of e.g. temperature, pH, enzyme activity, carbon dioxide tension, oxygen tension, enzyme activity, ion concentration, tissue water diffusion, pressure, tissue, electrical activity. More specifically, the membrane or matrix permeability should not increase in response to normal or pathological physiological conditions in mammals, moreover, the matrix or membrane should not suffer chemical or physical breakdown vis-à-vis said the mentioned conditions. In positive terms, the matrix or membrane material is responsive only to non-physiological parameters and the response is chemical or physical breakdown of the matrix or membrane material, to cause the relaxation efficiency of the internal T1 agent to increase. This to ensure that the drug load is not released uncontrolled, but always in response to an extra-corporal stimuli, like e.g. light or ultrasound.

The membrane or matrix material may form a functionalized cubic gel precursor, functionalized cubic liquid crystalline gel, a dispersion of functionalized cubic gel particles, a functionalized cubic gel particle, gel, precursor, dispersion. It may also form a polymer-based, alginate or chitosan nanoparticle. In a preferred embodiment of the current invention the membrane or matrix material is a phospholipid membrane, forming a liposome. The gel-to-liquid crystalline phasegeltransition temperature (Tc) of the liposome membrane must be higher than normal or pathological physiological temperatures, that is, under no circumstances lower than 42° C.

A liposomal product for parenteral administration demands high chemical and colloidal stability both during storage and use. Additionally, it must be non-toxic and biologically compatible, e.g. isotonic and isohydric. The composition and design of the liposome depend upon the properties and applications of the liposomal product. Charge stabilization of liposomes is achieved by imparting a surface charge to the liposome surface, which is accomplished by employing negatively or positively charged phospholipids. Polymeric coating materials, such as polyethylene glycol (PEG), are also used to prevent particle fusion or aggregation by steric hindrance. Liposomes of high chemical and colloidal stability are normally obtained by saturated phospholipids with a gel-to-liquid crystal phase transition temperature (Tc) above 42° C., in practice phospholipids having saturated fatty acid portions with an acyl chain length of 14 carbon atoms or more are used. This is a crucial feature for liposome encapsulated material as the risk of leakage during storage and also in vivo is minimized. For membrane incorporated material, the use of saturated phospholipids is not so critical is for minimizing leakage; however the use of saturated phospholipids is preferred to achieve acceptable chemical stability.

The membrane composition chosen will result in liposomes that are physicochemically robust and that retain incorporated or encapsulated material both during extended storage and in vivo. A sterol component could be included to confer suitable physicochemical and biological behavior. The sterol component in the liposomes of the present invention is suitably cholesterol or its derivatives, e.g., ergosterol or cholesterolhemisuccinate, but is preferably cholesterol. The sterol should be present in an amount that enables maximum retention of entrapped or incorporated material, minimizes alterations in physicochemical properties (e.g., liposome size and size distribution) during long-term storage but without negatively affecting the conditions of exchange limitations prior to chemical or physical breakdown of the membrane material. Calcidiol or calcidiol derivates may also be used conveying both structural and therapeutic advantages.

The membrane bilayer of the liposomes of the present invention preferably contains negatively charged and neutral phospholipid components in such a combination or mixture that results in an overall Tc above 42° C. Typically, the selected phospholipids will have saturated fatty acid portions with an acyl chain length between 14 and 20 carbon atoms. The neutral phospholipid component of the lipid bilayer is preferably a phosphatidylcholine, most preferably chosen from diarachidoylphosphatidylcholine (DAPC), hydrogenated egg phosphatidylcholine (HEPC), hydrogenated soya phosphatidylcholine (HSPC), distearoylphosphatidylcholine (DSPC), dipalmitoylphosphatidylcholine (DPPC) and dimyristoylphosphatidylcholine (DMPC). The negatively charged phospholipid component of the lipid bilayer may be a phosphatidylglycerol, phosphatidylserine, phosphatidylinositol, phosphatidic acid or phosphatidylethanolamine compound.

Liposomes of the present invention may be prepared by methods that are broadly known in the art (See Lasic, 1993)

The matrix or membrane material of the current invention may comprise photosensitizers, preferably photosensitizers based on the porphyrin skeleton, particularly disulfonated tetraphenylporphine (TPPS2a) or aluminium phthalocyanine (AIPcS2a). These photosensitizers render possible drug release by means of light, acoustic energy or cavitation.

Furthermore the particulate material may comprise an air bubble, e.g. a liposome comprising air bubbles like perfluorobutane, to increase the ultrasound sensitivity. However, an air bubble will typically not be present. Microbubbles, that is, phospholipid encapsulated air bubbles, are not part of the current invention.

The particulate material may be sensitive to high temperatures, light of defined wavelength, cavitational effects, exogenously generated acoustic energy to induce drug release. High temperatures herein means above normal and pathological physiological levels, typically above 42° C. In a preferred embodiment of the current invention the particulate drug delivery material or matrix or membrane material, e.g. the liposome, is sensitive to acoustic energy, more particularly, ultrasound. An ultrasound sensitive material in the context of drug release means a material responding to ultrasound or acoustic energy by releasing its drug contents. The particular mechanism of release is not relevant, however, relaxation exchange limitations must be suspended during and/or directly after drug release. This typically means disrupting or breaking down the membrane to a degree dramatically increasing the T1 relaxation efficiency of the internal T1 contrast generating species. The ultrasound waves may be of any frequency or amplitude provided that said ultrasound waves induce drug release from the particulate material of the invention. It is, however, preferred that the chosen frequency and amplitude induce cavitation. More particularly, it is preferred that the frequencies are below 1.5 MHz, more preferably below 1.1 MHz. In preferred embodiments the frequency is 1 MHz, 500 kHz, 40 kHz or 20 kHz.

The diameter of the particulate material should not exceed 1000 nm. Preferably the diameter is below 250 nm, more preferably below 150 nm, and even more preferably around 100 nm, e.g. with the liposome population diameter peak within the range 80 nm to 120 nm. Such a small size is preferred to maximize the probability of passive accumulation in target tissue due to the Enhanced Permeability and Retention Effect (EPRE) (Matsumura et al. 1989).

The drug encapsulated by the current particulate material may be of any suitable chemical or therapeutic type. It is, however, preferred that the drug is hydrophilic or amphiphilic, more preferably hydrophilic. Given that the current invention is related to local release of drugs it is also implied that drugs used should benefit from local release by the current invention. Such drugs are typically anti-inflammatory drugs, antibiotics, anti-bacterial drugs, cardiovascular drugs or anti-cancer drugs. In a preferred embodiment of the current invention the drug is an anti-cancer drug. The particle of the invention may also be designed to incorporate multiple drugs.

As mentioned above, the T1 relaxation efficiency of the internal T1 magnetic resonance contrast generating species varies in response to drug release, more specifically, the effect of the internal contrast species on the MR image is only visible during and/or after drug release. This is possible because drug release, particularly ultrasound induced drug release, will always coincide with relief of the relaxation exchange limitations and increased water accessibility. The internal species is a T1 magnetic resonance contrast agent of any type known to a skilled person, see e.g. EP 1069 88 B1. Typically, gadolinium chelates and manganese compounds are used. One or several T1 agent species may be comprised in the drug delivery particle, however one species is preferred. ‘Internal’ in the current context means not exposed to bulk water until the point of drug release. If the drug delivery particle is a liposome this means within the aqueous interior of the liposome, attached to the inner surface of the liposomal membrane or comprised in the membrane shielded from bulk water. If the T1 contrast agent is attached on the inner side of a membrane or matrix, e.g. on the inner side of the liposomal membrane, it is important to minimize so-called anisotropic motions (Tilcock et al. 1992. Parac. Vogt et al. 2006). Association with a phospholipid membrane may be achieved by linking the contrast agent to a phospholipid or rendering the contrast agent amphiphilic. Linking to phospholipids, making amphiphiles, minimizing anisotropic motions and loading particles (e.g. liposomes) with contrast agents are all within the skills of the artisan.

In a preferred embodiment the internal T1 contrast agent is a Gd chelate encapsulated in the aqueous phase of the particulate carrier and/or attached to the inner surface of the particulate carrier membrane. The internal T1 agent distribution renders qualitative and/or quantitative monitoring of the drug release process possible.

The above-mentioned internal T1 agent should be comprised in the aqueous phase of the drug delivery particle, e.g. the liposome, if the drug is hydrophilic. On the other hand, if the drug is amphiphilic or lipophilic, then the T1 agent should be associated is with the inner surface of the particulate carrier membrane or comprised in the matrix or membrane material shielded from bulk water. Hence, the internal T1 agent should mimic the solubility properties of the drug in question. In a preferred embodiment, the T1 agent is a hydrophilic compound.

The external T1 magnetic resonance contrast generating species must, as described above, possess a high level relaxation efficiency before drug release to make determination of spatial position possible. In this way sufficient particle accumulation in the diseased volume, e.g. tumour, may be ensured before induction of drug release. Hence, the external magnetic resonance contrast generating species is a T1 agent of any suitable type known to a person skilled in the art, see e.g. EP 1069 88 B1. In addition, the external T1 agent must be associated or linked to the particulate material in a way exposing it to bulk water. In the case of a liposome drug carrier, the external T1 agent may be, e.g., a phospholipid associated Gd chelate. The external agent may also be a amphiphilic T1 agent with one lipophilic part anchored in the membrane or matrix material and the hydrophilic part containing the Gd chelate protruding into the bulk. In both cases it is important to minimize anisotropic motions to obtain optimal contrast efficiency. One or several external T1 agent species may be comprised in the drug delivery particle, however one species is preferred. Typically, gadolinium compounds are employed. In a preferred embodiment the T1 agent is an amphiphilic Gd chelate with a lipophilic side chain suitable for membrane incorporation.

In functional terms, the T1 effect of the external T1 magnetic resonance agent present in intact particles is exploited to monitor extent of particle accumulation in the diseased volume, whilst the T1 effect of the internal T1 agents is induced as a result of membrane or matrix breakdown making drug release monitoring possible.

Another aspect of the current invention is use of the particulate material described supra for the manufacture of a particulate drug delivery system for treating cancer, cardiovascular disease, immunological, infective, and inflammatory disease. The drug may be released from the particle by means of e.g. ultrasound, heat or radiation. Preferably, the drug is release by means of ultrasound.

A further aspect of the present invention is use of the particulate material of the invention for monitoring spatial position of said material before drug release and efficiency of drug release.

The present invention also comprises use of a particulate material comprising a matrix or membrane material, a drug, and at least one T1 magnetic resonance contrast generating species, said matrix or membrane material being responsive to a pre-selected physiological parameter and the response is chemical or physical breakdown of the matrix or membrane material, to cause the relaxation efficacy of said contrast generating species to vary in response to said parameter for the manufacture of a particulate drug delivery system for treating cancer, cardiovascular disease, immunological and inflammatory disease. Preferably, the internal and external T1 magnetic resonance contrast generating agents are of the same species.

Also, the current invention comprises use of a particulate material comprising a particulate material comprising a matrix or membrane material, a drug, and an internal T1 magnetic resonance contrast agent and an external T1 magnetic resonance contrast agent, wherein the internal T1 agent is shielded from bulk water for the manufacture of a particulate drug delivery system for treating cancer, cardiovascular disease, immunological and inflammatory disease. The drug may be released by means of acoustic energy.

Furthermore, the current invention comprises a method of monitoring drug release in a mammal comprising the steps of administering parenterally to said mammal the particulate drug delivery material of the present invention; generating T1 weighted image data of at least part of said body in which said material is present; and generating therefrom a signal indicative of the level of accumulation of said material; inducing drug release; generating new T1 weighted image data of at least part of said body in which said material is present; and generating therefrom a signal indicative of the level of drug release. The ‘level of drug release’ indicates the quantitative and/or qualitative level of release. T1 weighted images will also be accuired prior to parenteral administration of the particulate drug delivery material.

### DESCRIPTION OF THE DRAWINGS

FIG. 1. Relaxation mechanisms of particulate carrier (e.g. liposome) comprising an internal and external distribution of T1 agent per se prior to (a) and, after (b) membrane or matrix breakdown

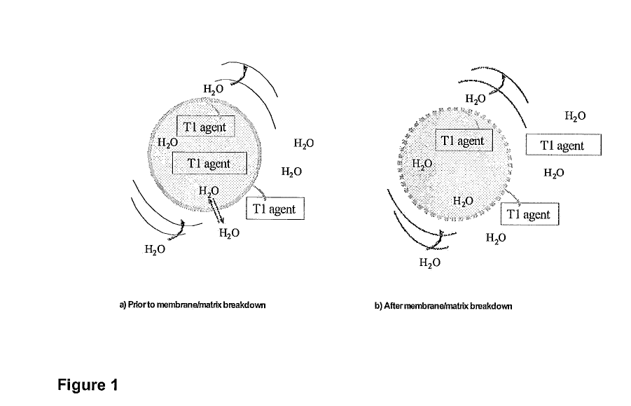

a)
Susceptibility effect due to water diffusion through field gradients
Limited T1 effect of internal T1 agent encapsulated within aqueous interior or bound to inner membrane surface due to low water accessibility
High T1 effect of external T1 agent due to reduced motion and high water accessibility
=>Hyperintensity on T1 weighted MR images

b)
Internal encapsulated hydrophilic T1 agent released from particle
Susceptibility effect decreased
Unchanged high T1 effect of external T1 agent
Enhanced T1 effect of released T1 agent and inner membrane bound T1 agent due to higher water accessibility
Marked hyperintensity on T1-weighted MR images

FIG. 2. Schematic and simplified representation of a particulate T1 contrast switch where the T1 effect and, hence, signal intensity is increased as a result of membrane or matrix breakdown of the particulate carrier.

### EXAMPLES

The following examples are meant to illustrate how to make and use the invention. They are not intended to limit the scope of the invention in any manner or to any degree.

### Example 1

### Preparation and MR Evaluation of Liposome Containing Amphiphilic Gd Chelate

DSPC, DSPE-PEG 2000 and amphiphilic Gd chelate are dissolved in a chloroform/methanol mixture (volume ratio; 10:1) and the organic solution is evaporated to dryness under reduced pressure. Liposomes are formed by the film hydration method, by hydrating the lipid film with a pre-heated (65° C.) buffered sucrose solution. The liposomes are subjected to several freeze-thaw cycles and allowed to swell for two hours at a temperature above the Tc of the phospholipid mixture. The liposome dispersion is extruded at a temperature above the Tc of the phospholipid mixture through polycarbonate filters of various pore diameters to achieve a liposome size around 100 nm. Untrapped Gd chelate is removed by dialysis against isosmotic and isoprotic sucrose solution.

The in vitro MR imaging efficacy of the liposomes is investigated in a suitable gel phantom at clinically relevant field strengths. T1 weighted and T2 (T2*) weighted images of the phantom are acquired prior to and after liposome disruption, the latter achieved by ultrasound treatment.

### Example 2

### Preparation and MR Evaluation of Liposome Containing Both an Amphiphilic Chelate and a Water Soluble Gd Chelate

DSPC/DSPE-PEG 2000 liposomes containing both an amphiphilic Gd chelate and a water soluble Gd agent are prepared and purified analogously to Example 1, except that the buffered sucrose solution used for lipid film hydration also contains a water soluble Gd chelate.

The in vitro imaging efficacy of the liposomes is investigated in a gel phantom at clinically relevant field strengths as described in Example 1.

### Example 3

### Preparation and MR Evaluation of Liposome Containing an Amphiphilic Gd Chelate, a Water Soluble Gd Chelate and a Drug Marker

DSPC/DSPE-EPG 2000 liposomes containing an amphiphilic Gd chelate, a water soluble Gd chelate and a drug marker are prepared and purified analogously to Example 2, except that the buffered sucrose solution used for lipid film hydration also contains the fluorescent dye calcein.

The in vitro imaging efficacy of the liposomes is investigated in a gel phantom at clinically relevant field strengths as described in Example 1.

Having now fully described the present invention in some detail by way of illustration and example for purpose of clarity of understanding, it will be obvious to one of ordinary skill in the art that same can be performed by modifying or changing the invention by with a wide and equivalent range of conditions, formulations and other parameters thereof, and that such modifications or changes are intended to be encompassed within the scope of the appended claims.

### REFERENCES

Barsky et al. Theory of paramagnetic contrast agents in liposome systems. Magn Reson Med. 1992; 24:1-13.

Bednarski et al. In vivo target-specific delivery of macromolecular agents with MR-guided focused ultrasound. Radiology. 1997; 204: 263-268.

Caravan et al. Gadolinium(III) Chelates as MRI Contrast Agents: Structure, Dynamics, and Applications. Chem Rev. 1999; 99:2293-22352.

EP1069888B1 Fossheim et al. Use of particulate contrast agents in diagnostic imaging for studying physiological parameters.

Fossheim et al. Low molecular weight lanthanide contrast agents: in vitro studies of mechanisms of action. J Magn Reson Imaging. 1997; 7:251-7.

Fossheim et al. Paramagnetic liposomes as MRI contrast agents: influence of liposomal physicochemical properties on the in vitro relaxivity. Magn Reson Imaging. 1999a; 17:83-9.

Fossheim et al. Thermosensitive paramagnetic liposomes for temperature control during MRI guided hyperthermia—in vitro feasibility studies. Acad Radiol. 2000; 12:1107-1115.

Fossheim et al. Investigation of lanthanide-based starch particles as a model system for liver contrast agents. J Magn Reson Imaging. 1999b; 9:295-303.

Gløgård at al. Liposomes as carriers of amphiphilic gadolinium chelates: the effect of membrane composition on incorporation efficacy and in vitro relaxivity. Int J Pharm. 2002; 233:131-140.

Lasic D D. Preparation of liposomes. In: Lasic D D, ed. Liposomes from physics to applications. Amsterdam, The Netherlands: Elsevier Science Publishers B. V., 1993; 63-107).

Matsumura et al. A new concept for macromolecular therapeutics in cancer chemotherapy: mechanism of tumoritropic accumulation of proteins and the antitumor agent smancs. Cancer Res. 1986; 46:6387-6392.

Mulder et al. Lipid-based nanoparticles for contrast-enhanced MRI. NMR Biomed 2006; 19: 142-164.

Parac-Vogt et al. Paramagnetic liposomes containing amphiphilic bisamide derivatives of Gd-DTPA with aromatic side chain groups as possible contrast agents for magnetic resonance imaging. Eur Biophys J. 2006; 35:136-144.

Pitt et al. Ultrasonic drug delivery—a general review. Expert Opin Drug Deliv. 2004 November; 1:37-56.

Richardson et al. Pharmaceutical applications of magnetic resonance imaging (MRI). Adv Drug Deliv Rev. 2005; 57:1191-1209.

Rubesova et al. Gd-labeled liposomes for monitoring liposome-encapsulated chemotherapy: quantification of regional uptake in tumor and effect on drug delivery. Acad Radiol. 2002; 9 Suppl 2:S525-7.

Saito at al. Gadolinium-loaded liposomes allow for real-time magnetic resonance imaging of convection-enhanced delivery in the primate brain. Exp Neurol. 2005; 196:381-9.

Tilcock et al. Liposomal Gd-DTPA: preparation and characterization of relaxivity. Radiology. 1989; 171:77-80.

Tilcock et al. The design of liposomal paramagnetic MR agents: effect of vesicle size upon the relaxivity of surface-incorporated lipophilic chelates. Magn Reson Med. 1992; 27:44-51.

Tilcock. Delivery of contrast agents for magnetic resonance imaging, computed tomography, nuclear medicine and ultrasound. Adv Drug Deliv Rev. 1999; 37:33-51.

Viglianti et al. In vivo monitoring of tissue pharmacokintetics of liposome/drug using MRI: Illustration of targeted delivery. Magnetic Resonance in Medicine, 2004; 51:1153-1162.

1-11. (canceled)

12. A trackable particulate material for drug delivery comprising a matrix or membrane material, a drug, and an internal T1 magnetic resonance contrast agent and an external T1 magnetic resonance contrast agent, wherein the internal T1 agent is shielded from bulk water and the external T1 agent is exposed to bulk water, and wherein the T1 agent is a gadolinium and/or a manganese compound.

13. The particulate material of claim 12, wherein the matrix or membrane material is responsive only to non-physiological parameters and the response is chemical or physical breakdown of the matrix or membrane material, to cause the relaxation efficiency of the internal T1 agent to increase.

14. The particulate material of claim 12, wherein the matrix or membrane material is a phospholipid membrane.

15. The particulate material of claim 12, wherein said matrix or membrane material comprises a liposome.

16. The particulate material of claim 12, wherein the T1 agent is a gadolinium compound.

17. The particulate material of claim 12, wherein the T1 agents are comprised:
in the aqueous phase of a liposome and/or on the inner surface of a liposomal membrane; and
on the exterior surface of a liposomal membrane.

18. The particulate material of claim 12, wherein said material is for medical use.

19. A method of treating cancer, cardiovascular disease, immunological, infective and inflammatory disease comprising
administering the particulate material of claim 12 to a patient in need thereof

20. A method of monitoring drug release in a mammal comprising
administering parenterally to said mammal the particulate material of claim 12;
generating T1weighted image data of at least part of said body in which said material is present;
generating therefrom a signal indicative of the level of accumulation of said material;
inducing drug release;
generating new T1 weighted image data of at least part of said body in which said material is present; and
generating therefrom a signal indicative of the level of drug release.

21. A method for treating cancer, cardiovascular disease, immunological and inflammatory disease comprising
administering to a patient in need thereof a particulate material comprising a matrix or membrane material, a drug, and an internal T1 magnetic resonance contrast agent and an external T1 magnetic resonance contrast agent, wherein the internal T1 agent is shielded from bulk water.

22. The method of claim 21, wherein the drug is, released by means of acoustic energy.

23. The particulate material of claim 14, wherein said phospholipid membrane comprises a liposome.
